(19) United States
(12) Patent Application Publication
(10) Pub. No.: US 2008/0206406 A1
(43) Pub. Date: Aug. 28, 2008

(54) Modified Proteins With Altered Aggregation Properties

(75) Inventors: Harmen Henri Jacobus De Jongh, Zeist (NL); Hendrek Albertus Kosters, Ede (NL)

Correspondence Address: FOLEY AND LARDNER LLP;SUITE 500, 3000 K STREET NW, WASHINGTON, DC, 20007, US

(73) Assignee: CSM Nederland B.V.

(21) Appl. No.: 11/721,964

(22) PCT Filed: Dec. 15, 2005
(86) PCT No.: PCT/NL05/50077

§ 371 Date: Jan. 23, 2008

(30) Foreign Application Priority Data
Dec. 15, 2004 (EP) 04078407.6

(51) Int. Cl. A23J 3/16; C07K 1/107; C07K 2/00; A23L 2/66; A23L 1/304; A23J 3/08; A23C 9/152; A23C 9/13

(52) U.S. Cl. 426/74; 530/345; 530/322; 426/656; 426/657; 426/520; 426/580; 426/598

(57) ABSTRACT

The invention relates a method for preparing modified proteins having an increased capacity to bind divalent cations. For example the concentration of calcium at which Ca²⁺ induced aggregation occurs is increased by subjecting a protein to Maillard reaction conditions. Such modified proteins are of use for making calcium fortified food products.

### FIELD OF THE INVENTION

The present invention relates to the field of chemical protein modification and food products comprising such modified proteins. In particular, the invention relates to methods for making cation fortified food products, and food products comprising proteins or protein fragments with an increased capacity to accommodate dissolved cations.

### BACKGROUND OF THE INVENTION

Protein aggregation and factors affecting protein aggregation have been widely studied in the pharmaceutical industry and food industry. Protein aggregation is often triggered by factors such as elevated temperatures (heat treatment), pH and/or calcium-ion availability. Calcium ion availability has been described to influence aggregation of crude whey protein mixtures (Barbut and Foegeding 1993, J Food Sci 5:867-871; Haggett 1976, J Dairy Sci and Technol 11: 244-250; Ju and Kilara 1998, J Dairy Sci 81: 925-931; Morr and Josephson 1968, J Dairy Sci 51: 1349-1451; Sherwin and Foegeding 1997, Milchwissenschaft 52:93-96; Varunsatian et al. 1983, J Food Sci 48: 42-47; Zhu and Damodaran 1994, J Agric and Food Chem 42: 856-862) and has been shown to influence aggregation of β-lactoglobulin, which is a major protein component of whey (Simons et al. 2002, Arch Biochem Biophys 406(2): 143-152).

In food manufacturing processes protein aggregation is of major importance for the manufacturing process and the composition of the final product. It has been shown (Simons et al., Arch. Biochem. Biophys 2002 406(2):143-52) that the sensitivity to the presence of calcium for protein aggregation is directly related to the availability of carboxylate groups on the protein, as could be achieved by methylation or succinylation of proteins. However, alternative, more food-grade methods are desirable.

The calcium levels of products containing proteins which are sensitive to calcium-induced aggregation are kept low, in order to avoid protein aggregation or precipitation during manufacture or storage. This does however lead to products which are low in calcium, such as the soy-milk products, and may result in calcium deficiency in subjects, such as persons who cannot consume milk products due to milk allergy or lactose intolerance. Previous attempts to provide a stable soy milk having elevated calcium levels have resulted in coagulation and precipitation of soy protein via a protein-ionic calcium interaction.

Various chemicals have been employed to chelate calcium ions and prevent soy protein precipitation.

U.S. Pat. Nos. 1,210,667 and 1,265,227 teach beverages containing sodium phosphate as the chelating agent for calcium ions. Weingartner, et al proposes calcium citrate as a cheating agent (J Food Sci. 256-263(1983)). Hirotsuka, et al proposes a process which employs sonication of lecithin in a solution containing EDTA to envelope the calcium ions present in solution (J. Food Sci. 1111-1127 (1984)). EP 0195167 discloses the addition of polyphosphate to soy milk which increases calcium binding without precipitating soy protein-calcium complexes. WO 03/053995 teaches that phosphorylation of soybean protein followed by hydrolysis and calcium-binding reaction leads to high calcium-binding ability of the protein in combination with good water-solubility.

Several of the chelating agents previously employed reduce the bioavailability of the calcium ions in solution in the milk. Thus, while total calcium ion concentration in the milk may be increased over unfortified soy milk, a large portion of the added calcium remains nutritionally unavailable.

Besides soy milk, numerous other food products would benefit from calcium enrichment. For example, animal milk products (particularly those formed from cow's milk) are already considered to be a good dietary source of calcium. However, these products contain only limited quantities of calcium in each serving, requiring the average person to consume a large portion of the product to obtain the recommended daily allowance (RDA) of calcium. Furthermore, some people have medical conditions (e. g., osteoporosis) which require the consumption of calcium beyond that required for other people. Therefore, supplemental products which increase the amount of calcium in each serving of milk products and without negatively affecting the quality of the milk product are always in demand.

Healthy nutrition should provide, besides calcium, also other essential elements. In particular in view of calcium fortification it is of interest to regard the amount of magnesium in food products in order to keep the calcium/magnesium ratio in balance.

Thus, it is desirable to provide a method for fortifying food products, in particular milk based products, e.g. cow's milk and soy milk bases products, with cations, in particular calcium and magnesium, without coagulation of the proteins and cations. It is further desirable to employ a method to prevent coagulation that avoids the use of reagents that reduce the bioavailability of the cations in solution in the milk based products and that thus provides minimal decrease in the bioavailability of the cations present in the food products.

### DESCRIPTION OF THE INVENTION

The present inventors found that subjecting proteins to Maillard reaction conditions leads to a decrease in the protein's sensitivity to calcium-ion (Ca²⁺) induced protein aggregation. In other words, Maillardated proteins remain in solution while the concentration of dissolved calcium increases. In a Maillard reaction basically the reducing end of a sugar reacts with a primary amine group. In particular the lysine residues of the soy protein glycinin (11S globulin) and of soybean protein isolate (SPI) were modified by controlled Maillardation, resulting in a significant decrease in calcium induced aggregation. Even better results were obtained in case of whey protein being modified by controlled Maillardation. Effectively, the controlled Maillardation results in protein products of which the lysine residues are glycosylated. Without being bound by theory it may be so that modification of lysine residues results in a ‘liberation’ of a previously ionically paired carboxylate on the protein surface. Based on this finding it is possible to manufacture products with higher levels of cations such as calcium and magnesium, as the modified proteins increase the threshold level at which cation-induced protein aggregation occurs. Because no chelating agents are introduced by Maillardation the bioavailability of the calcium and/or magnesium is not negatively influenced.

It is important to realise that modification of nutritional proteins should not lead to loss in functionality. For instance succinylation of a protein often very rapidly, already upon introduction of 2-3 succinyl groups per protein, may lead to a decrease of conformational stability. Concomitant with the loss of the native structure, the functionality of the protein is lost. However, for example all 16 lysine residues of β-lactoglobulin, the main constituent of whey, can be glucosylated under Maillard reaction conditions without showing a loss of the molecular native structure. Thus, advantageously, Maillardation in general does not impair this structural integrity of the modified protein.

Thus the present invention concerns a method for increasing the cation binding capability of a protein, said method comprising subjecting the protein to Maillard reaction conditions. In other words, the invention concerns the preparation of a modified protein that is capable of increased cation binding compared to non-modified protein said method comprising subjecting the non-modified protein to Maillard reaction conditions. The increase in cation binding capability should be such that upon increasing the concentration of cations aggregation of the protein does not occur. In one embodiment of this invention cation and cations refer to divalent cation or divalent cations. In a preferred embodiment cation and cations refer to Ca²⁺ and/or Mg²⁺.

In general the Maillard reaction can be described as gently heating sugars and amino acids in water. In the context of this invention Maillard reaction conditions means reaction of a protein of interest with a compound that comprises a reducing carbonyl moiety, in particular a carbonyl moiety that can react with a primary amine group in the protein of interest to form a Schiff base. Usually the primary amine group in a protein of interest is the amine of a lysine residue. Preferably the compound that comprises a carbonyl moiety is a carbohydrate, which may be an aldose as well as a ketose. In one embodiment the carbohydrate is a monosaccharide with a reducing carbonyl group functionality. Examples of monosaccharides are glyceraldehyde, erythrose, threose, ribose, arabinose, xylose, lyxose, allose, altrose, mannose, glucose, gulose, idose, galactose, tallose, dihydroxyacetone, erythrulose, ribulose, xylulose, psicose, sorbose, tagatose and fructose. Disaccharides such as lactose and maltose and to a lesser extent sucrose, in view of its reducing capacity, or higher oligosaccharides may be used as well. Preferably a compound that comprises a reducing carbonyl moiety is selected from glucose and fructose.

Factors that are of influence for the Maillard reaction are temperature, the presence of water/moisture and pH.

Usually at least to start the Maillard reaction it is necessary to heat the reaction mixture. It may be so that after starting the reaction by providing a sufficient amount of heat, the reaction will continue at room temperature. However, as is shown in the examples, heating may also be continued. Care should be taken not to heat too excessively in order to prevent irreversible denaturation of the protein. Sufficient and appropriate heating depends on the protein of interest and the carbohydrate used to modify the protein. For the purpose of this invention the reaction mixture should be heated to at least 40° C. and preferably should not exceed a temperature that is 5° C. below the denaturation temperature of the protein in aqueous solution. To allow proper control of the Maillardation it is desirable not to use too high temperatures such as for example not to heat above 65° C.

Water is required to allow the Maillard reaction to proceed. Conveniently the water is present as moisture in the atmosphere and the reaction is carried out under humid conditions, suitably of at least 55% humidity. The reaction may also be carried out in aqueous solution, but this generally gives less reproducible results and is considered to result in a higher degree of denaturation of the protein.

Below pH 6 the Maillard reaction does not proceed. Preferably the reaction is carried out under near neutral or alkaline conditions. Preferably at a pH in the range of 6-9, more preferably in the range of 7-8.

Further the type of compound comprising a reducing carbonyl moiety that is used is of influence on the Maillard reaction. Different compounds will have different reducing capacities. In particular the reducing capacity of monosaccharides differs significantly; the higher the reducing capacity the faster the reaction takes place. Depending on for instance the desired degree of protein modification, or in other words Maillardation, and/or the reaction time that is available, the skilled man will be able to select a suitable carbohydrate. Carbohydrates with reducing capacity can be determined using the Luff reagens as described in the examples. Carbohydrates that test positive in the Luff assay thus are in one embodiment preferred. In particular it is preferred to use glucose or fructose.

As is shown in the examples, by varying the time of reaction, the degree of modification of the protein of interest may be varied as determined by the number of lysine residues that is modified. Depending on the type of protein of interest and the desired cation tolerance of that protein, it is a matter of routine experimentation for the skilled person, given a set of conditions in terms of temperature, presence of water and pH, to what degree, in other words for how long, the Maillard reaction should proceed. Typically, for glucose incubation times of 2-5 hours at 55° C. at pH 7 are sufficient to obtain suitable degrees of modification.

In a convenient manner to carry out the method of the invention a mixture, in a dry or solid state, of a protein of which the cation binding is to be increased, in other words a protein that is to be modified, and a compound comprising a reducing carbonyl moiety, is heated to a temperature of at least 40° C. under an atmosphere of at least 55% humidity. A dry mixture in this context does not mean it is necessarily water-free, but rather it means in the absence of solvent. Preferably a compound comprising a reducing carbonyl moiety is selected from glucose and fructose.

Preferably the protein is subjected to Maillard reaction conditions to such an extent that the protein-product is still able to display an enthalpic change of minimal 90% compared to that of non-Maillardated protein during heat-induced unfolding. Such standard calorimetry measurements are well within the ambit of one skilled in the art. Alternatively, or in addition thereto, the Stokes radius preferably should not be increased by more than 5% under ambient conditions. The Stokes radius can be determined in standard light scattering experiments which are also well within the ambit of one skilled in the art.

As mentioned before, effectively the controlled Maillard reaction results in protein products of which the lysine residues are glycosylated. Besides the Maillard reaction other synthetic methods to glycosylate lysine residues are known in the art such as described for instance in Christopher et al., 1980. Advances in Carbohydrate chem. Biochem. 37, 225-28, Caer et al., 1990, J. Agric. Food Chem. 38, 1700-1706, Colas et al., 1993, J. Agric. Food. Chem. 41, 1811-1815, Hattori et al., 1996, J. Food Sci. 61, 1171-1176.

Thus by the controlled Maillardation described above or by other synthetic methods to glycosylate lysine residues a modified protein product is provided. In the context of this invention a modified protein product is defined as a lysine rich protein of which at least 20%, preferably at least 30% and more preferably at least 40% of the lysine residues have been glycosylated. Lysine-rich is defined as a protein containing lysine residues, preferably at least 4.5 wt. % lysine per gram protein. These definitions relate to lysine residues that are available for glycosylation in the non-modified protein and can be determined by the OPA assay. Thus in one embodiment the invention concerns a modified protein product composition containing at least 80%, preferably at least 90%, more preferably at least 95% by weight of dry matter of glycosylated lysine-rich protein, said lysine-rich protein containing preferably at least 4.5 wt. % lysine per gram of said protein, wherein at least 20%, preferably at least 30% and more preferably at least 40% of the lysine residues present in the lysine-rich protein have been glycosylated. In one embodiment the modified protein product composition is in substantially dry form, such as the dryness that results after freeze drying.

In the context of this invention a modified protein is defined as a protein of which at least 20%, or at least 30% or at least 40% of the available lysine residues is modified, i.e. a number of lysine residues is no longer present as such, as compared to the non-modified protein as determined by the OPA assay, see examples. In other embodiments at least 45%, or at least 50% or at least 55%, for example up to 60% or 65% of the lysine residues is modified. Also a higher percentage of the lysine residues may be modified. It is preferred however that the structural integrity is not impaired and/or the functionality of the protein is not lost. It may be envisaged that due to the Maillardation the structural integrity is affected, but that during gastro-intestinal processing the modified protein retains or regains its functional (nutritional) properties, or at least a part thereof. While such a protein may not fulfil the criterion for enthalpic change during heat-induced unfolding as described above, it is to be understood that such a modified protein still falls under the scope of the present invention.

The OPA assay is suitable to determine the degree protein modification, in this particular case glycosylation, based on the specific reaction between ortho-phthaldialdehyde (OPA) and free primary amino groups in proteins and is essentially described by Church et al. (1983) Dairy Sci, 66, 1219-1227.

In short, the OPA-reagent is prepared by dissolving 40 mg OPA in 1 ml methanol, followed by the addition of 25 ml 0.1 M Borax buffer, 200 mg DMA and 5 ml 10% SDS. In the presence of 2-(dimethylamino)ethanethiol (DMA) primary amino groups in proteins react to alkyl-iso-indole derivatives that show absorbance at 340 nm. The volume is adjusted to 50 ml with demineralised water. A quartz cuvette is filled with 3 ml of this reagent and the absorbance at 340 nm is determined. Subsequently, 15 μl of a sample solution (protein concentration is determined by measuring the absorption at 280 nm, ε₂₈₀=0.712 ml*mg⁻¹*cm⁻¹) is added and after an incubation time of 30 min at room temperature, the absorbance at 340 nm is determined again. A calibration curve is obtained by adding 10, 20, 30, 40, 80, 100 and 150 μl of a 2 mM L-leucine solution in water to 3 ml of OPA-reagent, yielding concentrations in the range from 6.6 to 95.0 μM L-leucine. All measurements are performed at least in duplicate, preferably in triplicate.

Alternatively, or in addition to the percentage of lysine modification, a modified protein in the context of this invention is a protein which upon heating for 60 min at 95° C., preferably under an atmosphere of at least 55% humidity, results in browning of the protein. This browning can be quantified by measuring the absorbance at 514 nm. It is understood that a modified protein displays an absorbance of at least 0.10 absorbance units per cm light path at a concentration of 5 mg/ml.

Also, modification of the lysine residues in a protein leads to a change in isoelectric point (IEP) of the protein. Thus, alternatively or in addition to the percentage of lysine modification and/or the absorbance at 514 nm of the further heated product, in the context of this invention a modified protein is a protein which displays an IEP that is lower compared to the isoelectric point of the unmodified protein as can be determined by gel electrophoresis. Preferably the IEP is at least 0.2 pKa units lower, but not more than 1.0 pKa units.

Once the number of available lysine residues in a protein is known, for instance by using the OPA assay, the percentage of modification may also be determined by means of mass spectrometry, for instance MALDI-TOF MS. It may be expected that the increase in mass after modification correlates with a whole number of carbohydrate molecules that is used.

In one embodiment of the invention a modified protein product is provided, which has the ability to significantly increase cation tolerance of a food product when added to a food product in suitable amounts. In order to be applied in or added to a food product it may be necessary to purify and/or isolate the modified protein from the Maillard reaction. Purification and/or isolation can be carried out by conventional means known in the art such as dialysis, centrifugation, chromatography, crystallization, freeze drying, lyophilisation etc, as long as the material does not loose its functionality by the process. Thus the invention also concerns the use of lysine-rich protein wherein at least 20%, preferably at least 30% and more preferably at least 40% of the lysine residues have been glycosylated in the preparation of water-based cation-fortified food products in which the glycosylated lysine-rich protein is fully dissolved and in which the non-glycosylated form of the lysine-rich protein would precipitate as cation complexed protein salt, said lysine-rich protein containing preferably at least 4.5 wt % lysine per gram of said protein. In one embodiment the cation is a divalent cation. In specific embodiments the cation is Ca²⁺ or Mg²⁺ or a mixture of Ca²⁺ and Mg²⁺.

A “food” or “food product” or “foodstuff” is herein understood to refer to solid, semi-solid or liquid nutrient compositions or nutrient supplements, such as a drink e.g. dietary/health drinks and sports drinks and vitamin drinks, reconstituted milk, UHT milk, condensed milk, whey protein hydrolysates and isolates, yoghurt, dessert, sauces, etc. in the form of liquids e.g. including clinical nutrition for example for tube-feeding, gels, powders (for example milk formula), e.g. instant milk powders and infant milk powders, tablets, capsules, etc. Of particular interest are soy-based dairy products, in particular soy milk and products derived thereof. Of even more interest are whey protein-based dairy products and products derived thereof. Taking the desired increase in cation tolerance into account, a skilled person can readily determine what is a suitable amount of the modified protein ingredient to be used, in view of the cation induced aggregation properties of said modified protein ingredient.

In a further embodiment the invention concerns a cation fortified water based food product comprising at least 0.2% lysine-rich protein by weight of water, wherein the lysine-rich protein preferably contains at least 4.5 wt % lysine per gram of said protein and wherein at least 20%, preferably at least 30% and more preferably at least 40% of the lysine residues present in the lysine-rich protein have been glycosylated, said food product being essentially free of insoluble cation-complexed lysine-rich protein salt and containing an amount of dissolved cations that would cause the non-glycosylated form of the lysine-rich protein to precipitate as cation complexed protein salt. In one embodiment the cation is a divalent cation. In specific embodiments the cation is Ca²⁺ or Mg²⁺ or a mixture of Ca²⁺ and Mg²⁺.

In further embodiments the cation fortified water based food product comprises at least 0.4%, or at least 0.6%, or at least 0.8%, or at least 1.0%, or at least 1.5%, or at least 2.0%, or at least 2.5%, or at least 3%, or at least 3.5% or at least 4.0% up to 5.0% lysine-rich protein by weight of water.

Preferably the cation fortified water based food product contains an amount of dissolved Ca²⁺ ions that is at least 5%, preferably at least 10%, more preferably at least 15%, even more preferably at least 20% more than the Ca²⁺ concentration at which the non-modified protein would precipitate. Alternatively the cation fortified water based food product contains an amount of dissolved Ca²⁺ and Mg²⁺ ions that is at 5%, preferably at least 10%, more preferably at least 15%, even more preferably at least 20% more than the Ca²⁺ and Mg²⁺ concentration at which the non-modified protein would precipitate. In yet another alternative the cation fortified water based food product contains an amount of dissolved Mg²⁺ ions that is at least 5%, preferably at least 10%, more preferably at least 15%, even more preferably at least 20% more than the Mg²⁺ concentration at which the non-modified protein would precipitate. Alternatively, the increase in cations that a cation fortified water based food product according to the present invention can accommodate may be expressed in terms of ppm. For example a food product comprising 2 wt % lysine-rich protein wherein at least 20% of the lysine residues present in the lysine-rich protein have been glycosylated, preferably comprises 100 ppm more divalent cations, preferably at least 150 ppm more, more preferably at least 200 ppm more divalent cations, in particular Ca²⁺, Mg²⁺ and a combination of the two, than the concentration of divalent cations at which the non-modified protein would precipitate. The same is true for lysine-rich protein with higher levels of lysine modification such as lysine-rich protein wherein at least 30%, or at least 40% of the lysine residues present in the lysine-rich protein have been glycosylated.

In one embodiment the cation fortified water based food product comprises dissolved Ca²⁺ ions and/or Mg²⁺ ions in a concentration of at least 2%, more preferably at least 4% by weight of the lysine-rich protein. In yet another embodiment, in particular in the case of lysine rich proteins with higher degrees of modification, the cation fortified water based food product comprises dissolved Ca²⁺ ions and/or Mg²⁺ ions in a concentration of at least 5%, or at least 6% by, or even at least 7% or more than 8% by weight of the lysine-rich protein. In an advantageous embodiment the cation fortified water based food product comprises dissolved Ca²⁺ ions and Mg²⁺ . In this embodiment it is of benefit to include Ca²⁺ ions and Mg²⁺ in a molar ratio Ca²⁺:Mg²⁺ in the range of from 1:1 to 6:1, preferably 2:1 to 4:1, for example in a molar ratio of about 3:1

In one embodiment the cation fortified water based food product contains at least 50 wt. %, preferably at least 80 wt. % water. At least 20%, preferably at least 30% and more preferably at least 40% of the lysine residues in the lysine-rich protein have been modified as compared to the non-modified protein as determined by the OPA assay. For example at least 45% or 50% or at least 55% up to 60% or 65% of the lysine residues have been modified. A further embodiment is wherein the lysine-rich protein exhibits an enthalpic change during denaturation that is at least 90% of its non-modified counterpart. In an embodiment the lysine-rich protein is soy protein, optionally selected from glycinin and soy protein isolate. In another embodiment the lysine-rich protein is whey protein.

In an alternative embodiment the invention concerns a cation fortified water based food product comprising at least 0.2% lysine-rich protein by total weight of the food product and at least 0.05% dissolved Ca²⁺ ions and/or Mg²⁺ ions by total weight of the food product, said food product being essentially free of insoluble cation-complexed lysine-rich protein salt, wherein the lysine-rich protein preferably contains at least 4.5 wt % lysine per gram of said protein and wherein at least 20%, preferably at least 30% and more preferably at least 40% of the lysine residues present in the lysine-rich protein have been glycosylated. Preferably the food product comprises at least 0.02 wt %, more preferably at least 0.04 wt % dissolved Ca²⁺ ions and/or Mg²⁺ ions by total weight of the food product. In one embodiment the food product even comprises at least 0.06 wt %, or at least 0.08 wt % dissolved Ca²⁺ ions and/or Mg²⁺ ions by total weight of the food product.

In one embodiment the cation fortified water product is cow's milk or products derived therefrom or other derived dairy products comprising whey protein, for example reconstituted milk, UHT milk, condensed milk, whey protein hydrolysates and isolates and again products containing whey protein hydrolysates and isolates, yoghurt, instant milk powders, infant milk powders etc. In one embodiment the calcium fortified water product is soy milk or products derived from soy milk.

The protein that is to be modified may be any protein, such as a protein isolated from natural sources, made synthetically or expressed using recombinant DNA technology and is lysine-rich as defined herein. Examples of proteins that are of use to be employed in cation fortified products are egg, whey, soy and pea proteins. In a preferred embodiment of the invention the protein is soy protein, in particular selected from soybean glycinin and soybean protein isolate or a combination thereof. Soy protein may inherently display problematic water-solubility behavior. Therefore it is preferred to use water-soluble soy protein. In another embodiment the protein is β-lactoglobulin or more general whey protein.

It is noted that in the context of this invention that the divalent cation, e.g. calcium is not considered to be the driving force of protein aggregation, but it is considered to be the inducer or trigger thereof. It is believed that the calcium, so to speak, shields charges of the protein, which results in species that experience less repulsive forces upon collision and/or allowing hydrophobic interactions. The same role may be ascribed to magnesium. Protein modification by Maillardation so to speak shields the protein form precipitation in the presence of what would otherwise be an excess or surplus of calcium and/or magnesium in the presence of unmodified protein.

Notwithstanding the possible negative effects of Maillardation on color, aroma and flavor of food products, the method of the present invention does not give rise to detrimental effects that cannot be overcome with natural or artificial aroma's.

### EXAMPLES

### Determination of Free Primary Amino Groups by OPA

Principle:

To determine the degree of lysine modification the method described by Church et al. (1983) Dairy Sci, 66, 1219-1227 can be used. This method is based on the specific reaction between ortho-phtaldialdehyde (OPA) and free primary amino groups in proteins in the presence of 2-(dimethylamino)ethanethiol hydrochloride (DMA), to give alkyl-iso-indole derivatives that show an absorbance at 340 nm. The determination is very specific for lysines and the protein N-terminal group over arginines.

Materials:

0.1 M Borax: Dissolve 19.07 gram di-Natriumtetraborat-Decahydrat (Na₂B₄O₇.10H₂O) in 500 ml milliQ water.

10% SDS: Dissolve 10 gram Sodiumdodecylsulfate in 100 ml MilliQ water. (wear a fine dust mask).

OPA reagens: The OPA-reagent is prepared by dissolving 40 mg OPA (Sigma, P-0657) in 1 ml methanol, followed by the addition of 25 ml 0.1 M Borax buffer, 200 mg DMA (Aldrich, D14.100-3) and 5 ml 10% SDS. Adjust the volume to 50 ml with MilliQ water.

2 mM L-Leucine Dissolve 13.1 mg L-Leucine (Pierce, Mw. 131.18) in 50 ml MilliQ water. Calculate the exact concentration.

Procedure:

Calibration Curve:

1: Fill six quartz cuvettes with 3 ml of OPA reagent, weight and determine the absorbance (Ablanc) at 340 nm.

2: Add 10, 20, 40, 80, 120 and 150 μl of the 2 mM L-leucine stock solution (weight all) to 3 ml of OPA-reagent, yielding concentrations in the range from 0.0066 to 0.095 mM L-leucine.

3: Incubate 10 minutes at room temperature.

4: Determine the absorbance at 340 nm.

5: Fit data by linear regression. Calculate the molar extinction coefficient (ε) of alkyl-iso-indole derivative (=slope of linear function, should be 7000±500 M⁻¹*cm⁻¹).

Sample Measurement:

1: Fill quartz cuvettes with 3 ml of OPA reagent, weight and determine the absorbance (Λ_{blanc}) at 340 nm.

2: Add 50 μl of a 5 mM NH₂-solution (approx. 10 mg/ml protein solution), weight and mix.

3: Incubate for 10 min at room temperature.

4: Determine the absorbance at 340 nm (Aₛₐₘₚₗₑ).

5: Calculate the molar concentration of NH₂: ΔA340/ε

6: Determine the molar protein concentration spectrophotometrically at 280 nm.

7: Calculate the amount of free NH₂ groups on the protein by dividing the molar concentration of NH₂ by the molar protein concentration.

Remark:

All measurements are performed at least in duplicate.

If OPA reagents turns into pink color instead of yellow before adding protein, most probably DMA is contaminated by secondary amines.

Soy Proteins

Soybean Glycinin

Soybean glycinin (ca. 12 grams; PR004, see below) was dialysed extensively (4×12 L milliQ) at 4° C. (end volumes ˜235 mL). The pH of the light brown/beige protein solution was adjusted to pH 8.0 with 0.1 M NaOH. Aliquots (˜58 mL; ca. 3 grams) was taken as control and to the rest of the protein solution (ca. 9 grams) fructose (3.3 grams, Merck reinst) was added and the solution was split in three ˜58 mL portions. After freeze drying the brownish crispy protein flakes were heated (55° C.) in an incubator containing a saturated NaNO₂ to assure 65% humidity throughout the Maillard reaction. After 2, 5 and 26 hours glycinin samples were cooled to 4° C. All protein samples were dissolved in milliQ (60 mL; when needed the samples were brought to pH 8.0 to dissolve the protein), dialysed against milliQ (4×12 L), freeze dried and stored at −20° C. until use.

The modification of the lysine after the Maillardation was determined with the OPA assay (WCFS Protocols Issued by B-009/B-010, AP12_{—}1, page 30, see also hereinbelow). The control was defined as 0% modification. After 2 hours˜3%, after 5 hours˜14% and after 26 hours˜54% of the available lysines were modified in glycinin.

The (modified) glycinins were dissolved (2 mg/mL) in 20 mM BisTris pH 7.0 and 20 mM Tris/HCl pH 8.0 by head-over-head agitation. At ambient temperature (˜22° C.) the calcium-dependent aggregation was measured at 540 nm in 1 mL disposable cuvettes. To the protein solutions aliquots of 100 mM CaCl₂ solutions in the respective buffers was added.

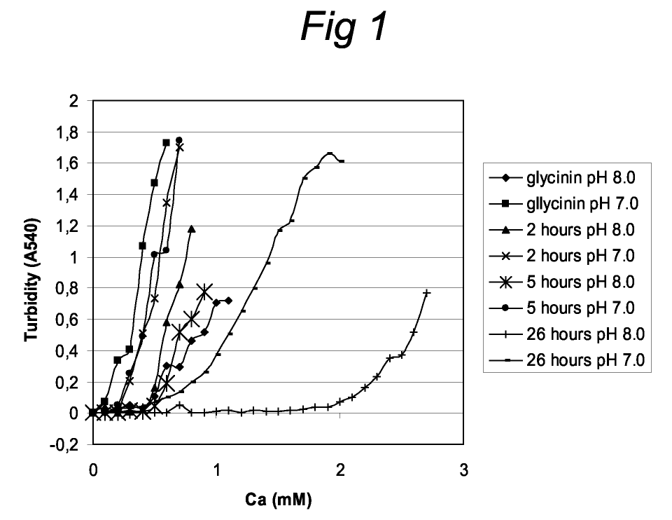

The calcium-triggered glycinin aggregation is shown in FIG. 1.

Soybean Protein Isolate

Soybean Protein Isolate (12 grams, SPI, see below, tube 4 in 200 mL 20% glycerol) was dialyzed (3×12 L) against demi water at room temperature. The pH of the water was adjusted to pH 8.0 with 1 M sodium hydroxide. To the dialysate 4.4 grams fructose (Merck, reinst) was added, the turbid brownish protein solution was frozen and freeze dried. The dried protein was put in an incubator at 55° C. above saturated sodium nitrate to assure 65% humidity. Samples (3 grams each) were taken after 3, 6 and 24 hours. The samples were dialyzed against demi water (3×12 L) at 4° C. and were subsequently freeze dried.

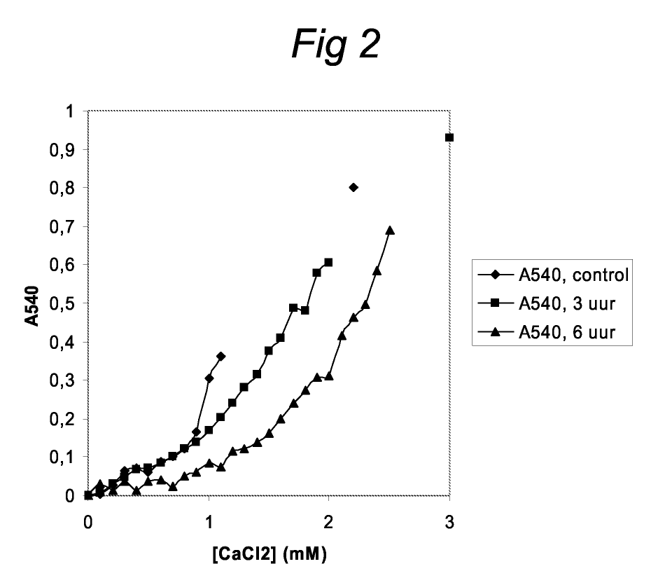

The SPI samples were dissolved (2 mg/mL) in 20 mM Tris/HCl pH 8.5. At lower pH the proteins were insoluble. At pH 8.5 the 24 hours sample was insoluble and the pH was adjusted to pH 10 to dissolve all material. In the latter preparation no Ca-induced aggregation was observed, even at [CaCl₂] of 30 mM. Because the aggregation may be affected by the pH, these data were not included in FIG. 2. The degree of modification of the SPI preparations was not determined.

The calcium-triggered SPI aggregation is shown in FIG. 2.

Storage Test with SPI

SPI-samples were subjected to a storage test. The results of the storage tests indicate that there are hardly any significant changes between the number of free primary amino groups prior and after the storage period. This means that the Maillard products in soluble state are stable for at least a weak at 4° C.

Purification of Soy Protein Isolates, Isolated Soy glycinin and Glycinin-Depleted Soy Protein Isolate from Whole Soybeans

One specific soy species was chosen (Williams '82; 1994 harvest) to ensure reproducibility of the composition of the isolated protein-fractions. The soybeans have been stored at −40° C. and defrosted directly prior to use. Thanh, V. H., and Shibasaki K. (1976), J. Agric. Food Chem. 24, 1117-1121 have described the principles of the fractionation, but the procedure has been adapted at various stages. Below a detailed description is given of the isolation procedure of the batches soy protein isolate (PR001), isolated soy glycinin (PR002), glycinin-depleted soy protein isolate (PR003) and isolated soy glycinin (PR004).

Description of the Method:

1: 60 kg whole soybeans were broken at 20° C. using a flake roller and the shells were removed using an air-sifter.

2: The broken beans were waltzed and milled at 4° C.

3: Next, the soy meal was packed on a stainless steel column in two aliquots of ±25 kg each (dimensions of column: ±30×150 cm) and 3 times 60 L of hexane was flushed repeatedly through the column at 10-15° C. to extract oil and fat. Next, the soy meal was dried during 24 hours to the air at 10-15° C.

4: The defatted soy meal (43 kg) was extracted with 575 L 30 mM Tris/HCl (pH 8.0) containing 10 mM 2-mercaptoethanol under continuous stirring at 10° C. for 1.5 hours. Using 4M NaOH the pH was continuously kept at pH 8.0. The suspension is stored overnight at 4° C. without stirring. Next, the suspension was centrifuged at 9000 g in a water-cooled continuous centrifuge set-up (Westfalia Separator AG, type BKAS-85-076) at <15° C. This was carried out in two aliquots of 8 and 35 kg subsequently, and the supernatants were combined.

5: The pH of the supernatant (protein extract) was lowered to pH 4.8 using 6M HCl, and the material was stirred for 2 hours at 4° C. Next, the pelleted material was removed by centrifugation as described above. In total 18.8 kg of material was pelleted.

Approximately 2 kg of this material was dissolved at 4° C. in 8.5 L 10 mM phosphate-buffer (pH 7.8) in the presence of 10 mM 2-mercaptoethanol and 20% glycerol and stored in 17 aliquots of 500 ml at −40° C. This sample is denoted as soy protein isolate (batch PR001, ±519 g protein/8500 ml).

6: 12.4 kg of the pellet obtained under 5 was dissolved in 120 L 10 mM phosphate-buffer (pH 7.8) in the presence of 10 mM 2-mercaptoethanol at 4° C. under 2 hours of stirring at 4° C. Next, the pH of the solution was lowered to pH 6.4 using 6 M HCl and the material was stirred for another 2 hours at 4° C. The pellet was again collected using a continuous centrifuge (9000 g).

7: The pellet inside the rotor (approximately 3.5 L of material) was suspended in 12 L 10 mM phosphate-buffer (pH 7.8) in the presence of 10 mM 2-mercaptoethanol at 4° C. . Ammoniumsulfate was added to obtain a saturation-percentage of 50%. After 1 hour of stirring at 4° C. the suspension was centrifuged (Sorvall GSA rotor, 30 min at 12000 g). Subsequently, again ammoniumsulfate was added to the supernatant to reach a saturation-level of 70% and after 1 hour of stirring the suspension was again centrifuged (Sorvall GSA rotor, 30 min at 12000 g) and the pellet was collected.

This pellet was dissolved in 1 L 10 mM phosphate-buffer (pH 7.8) at 4° C. in the presence of 10 mM 2-mercaptoethanol and 20% glycerol and stored in 4 aliquots of 250 ml at −40° C. This sample is denoted as isolated soy glycinin (batch PR002, ±56 g protein/1000 ml).

8: The remaining 4.4 kg of the pellet obtained under 5 was combined with all other pellet-fractions obtained under step 6 and dissolved in 120 L 10 mM phosphate-buffer (pH 7.8) in the presence of 10 mM 2-mercaptoethanol. The pH of the solution was now lowered to 6.0 (instead of the 6.4 as in step 6) using 6 M HCl and incubated for 2 hours at 4° C. under continuous stirring. The precipitated material was collected after the same continuous centrifuge-step as described above, the pellet appeared this time to be more solid.

9: The pellet (approximately 4 kg) was dissolved in 16 L 10 mM phosphate-buffer (pH 7.8) in the presence of 10 mM 2-mercaptoethanol at 4° C. and to this solution ammoniumsulfate was added up to a saturation-percentage of 45%. After 1 hour incubation at 4° C. the suspension was centrifuged (Sorvall GSA rotor, 30 min at 12000 g) and additional ammoniumsulfate was added to the supernatant up to a saturation of 75%. After incubation for 1 hour at 4° C. the material was again centrifuged (Sorvall GSA rotor, 30 min at 12000 g) and the pellet was collected.

This pellet was dissolved in 3.5 L 10 mM phosphate-buffer (pH 7.8) at 4° C. in the presence of 10 mM 2-mercaptoethanol and 20% glycerol and stored in 7 aliquots of 500 ml at −40° C. This sample is denoted as isolated soy glycinin (batch PR004, ±472.5 g protein/3500 ml).

10: The pH of the approximately 120 L of supernatant obtained during step 8 was lowered from pH 6.0 to 4.8 and incubated for 72 hours at 4° C. without stirring.

Next, the clear solution on top was gently removed and the precipitated material (3.5 L) was stored at −40° C. The suspension was defrosted and centrifuged (Beckman JA14, 30 min, 4° C. at 18000 g). The pellet was collected and dissolved in 10 mM phosphate-buffer pH 7.6 during 4 hours while the pH was kept at 7.6 with 2N NaOH.

This pellet was dissolved in 2.1 L 10 mM phosphate-buffer (pH 7.6) in the presence of 20% glycerol and stored in 44 aliquots of ca 50 ml at −40° C. This latter material is denoted as glycinin-depleted soy protein isolate (batch PR003, 150.4 g protein/2191 ml g).

Whey Protein

Materials

Experiments were carried out with Whey Protein Isolate (Bipro® Davisco) and Hiprotal 580G (kindly provided by Friesland Foods).

Maillardation

27 grams of whey protein isolate (WPI; Bipro) was dialysed against demi water. A sample (corresponding to 1/9 part of the total protein, i.e. 3 grams) was taken as a control, and the rest of the protein was split into two parts. Fructose was added to the one half, glucose to the other (4.5 grams) of the protein and the pH was adjusted to pH 5 or pH 8. Thereafter the mixtures were freeze dried.

15 g of Hiprotal was dialysed against 5 mM Tris-HCl pH 8.0 at 4° C. 5.3 g glucose was added to 423 ml of the dialysate. The pH of the dialysate was brought to pH 8, which was followed by freeze drying of the protein.

Maillard Reaction

The samples were subjected to Maillardation, as follows: The freeze-dried protein-carbohydrate mixtures were incubated at 50° C. and at 65% relative humidity in a Weiss cabinet. Samples were taken (0, 1, 2, 5, 8 and/or 24 hours) and stored at −20° C. until use. Thereafter, the products were re-dissolved in and dialysed against demi water. This is followed by freeze-drying and storage until use (4 or −20° C.).

OPA Assay

The degree of modification was determined by means of the OPA assay.

Ca-Induced Aggregation Studies

In order to determine the calcium-induced aggregation characteristics of the Maillard products, 10 mg/ml of the sample was dissolved in 10 mM Hepes pH 6.7+50 mM. The turbidity of the sample after heating (60-75° C.; length of the heating steps is indicated in the respective experiments) was checked spectrophotometrically at 500 nm as a function of the added calcium concentration. This was performed as an end point measurement (Pharmacia Ultrospec 4000) for all preparations. Kinetics studies of the aggregation were performed with the wheybased Maillard products by using a Varian Cary (10 mg/ml solutions in 10 mM Hepes pH 6.7+50 mM NaCl).

Shelf Life Test

A simplified shelf life test was performed with the Maillard products in solution. SPI and WPI: 10-40 mg/ml of the Maillard product was dissolved in 50 mM Tris-HCl of pH 7 or in succinic acid buffer at pH 4.5. Different amounts of CaCl2 were added to the samples and 50 mM NaCl was present in all samples. The OPA assay was performed prior and after storage (4° C.) of the samples to determine the degree of Maillardation. Thereafter, the samples heating test (analogous to Pasteurisation; short period of time at 75° C.) in order to determine the heat stability/Ca-induced aggregation of the samples. A comparable test was performed with SPI-W and Hiprotal. In this case the pH was 6.7 (50 mM Hepes+50 mM NaCl) at a temperature of 4° C. Additionally, a test at pH 8 (50 mM Tris-HCl+50 mM NaCl) and a storage temperature of 30° C.

Results

WPI (Bipro)

Maillardation of WPI proceeded with faster kinetics to a large degree of modification in the presence of glucose compared to fructose. For glucose after 8 hours of incubation about 20% of the available lysines were modified, after 24 hours more than 80%.

Calcium-Induced Aggregation of Bipro

From end point measurements, we learned that Maillard treated WPI (with glucose; 24 hrs) possessed significantly lower sensitivity to 50 mM calcium at 60° C. in comparison to the untreated sample. Samples with a higher degree of Maillardation, especially the WPI samples with glucose, incubated for 24 hours, have a low susceptibility to calcium induced aggregation. The calcium at which maximal aggregation occurs shifts to higher CaCl₂ concentration (from approx 10 to 25 mM).

Maillard products of WPI with glucose, incubated for 24 hrs, appear to be insensitive to CaCl₂ concentrations of up to 100 mM during heating at 60° C. Also in the case that the experiment was carried out at 70° C., a significant decrease of calcium-induced aggregation is seen in the WPI/glucose/24 hrs Maillard products compared to the non-Maillardated control.

Storage Test with Maillard Products from WPI

A storage test (pH 7, 4° C.) was carried out with a selection of WPI Maillard products. The samples contained various amounts, up to 100 mM, of CaCl₂. The calcium was added in order to see if it had a stabilizing effect on the Maillard products. The samples were stored and analysed by means of the OPA assay after one week.

It was shown that the 24 hours sample is stable in solution for at least a week. The presence of CaCl₂ does not seem to influence the stability of the Maillard product. Similar trends were observed in the storage test at pH 4. Also after 3 weeks, the Maillard products were still abundantly present in the samples.

Hiprotal 580G

To confirm the results found for Bipro a second series was performed using Hiprotal. One sample was given a much longer incubation time than the samples produced until now and was kept in the Weiss cabinet for Maillardation for 4 days (3 days at 50° C./65% relative humidity; followed by 1 day 55° C./65% RH). The sample was not dialysed after the Maillardation, so unbound glucose was still present, and was not subjected to the OPA assay in order to determine the amount of free primary amino groups. The sample is indicated as Hiprotal ‘long’.

Determining the free primary amino groups present in the Hiprotal samples by the OPA assay showed less decrease (lower degree of Maillardation) of the amount of primary amino groups in the protein samples compared to Bipro. After 8 hours of incubation about 20% of the available lysines were Maillardated, while after 24 hours about 40% had reacted.

Pasteurization Experiment with Hiprotal

In the case of Hiprotal/glucose 24 hours, the maximum of the turbidity curve is shifted to 50 mM calcium chloride. The Hiprotal ‘long’ sample showed to be hardly sensitive to CaCl₂.

Kinetic Measurements of Ca-Triggered Aggregation

The Maillard products of Hiprotal were subjected to kinetic calcium-dependent aggregation studies. The Hiprotal 24 hrs sample showed hardly any aggregation at 60° C., this is a significant decrease in the sensitivity towards calcium than in the case of the control. The Hiprotal 8 hrs sample performed better than the control in the 60° C. experiment, the Hiprotal 24 hrs is the better performer, however. Finally, the most striking result was obtained with the Hiprotal ‘long’ sample. Kinetic experiments showed that at 75° C. hardly any turbidity could be measured. Also, there was no precipitation noticed in this sample, at any calcium concentration. In contrast, turbidity was observed at 75° C. in the control samples and (to some extent) in the t=8 hours sample.

What can be clearly observed in the 60° C. trials is that the maximal turbidity is reached at a higher calcium concentration in the t=8 and t=24 hours samples (50 mM) as compared to the control (25 mM). This is a further indication that the sensitivity toward calcium is decreased by Maillardation. The extensively Maillardated Hiprotal was shown not to display significant turbidity at 75° C. with calcium concentrations up to 100 mM.

Storage Test of Hiprotal

The Hiprotal Maillard products are stable at 4° C./pH 6.7 for at least a week.

Determination of Reducing Carbohydrates using the Luff Reagens

The below-described procedure can be used to identify the presence of reducing carbohydrates. The method is qualitative.

Luff Reagens

Solution: A 25 g coppersulfate (CuSO4.5H2O) in 100 ml demineralised water.
B 50 g citric acid in 50 ml demineralised water.
C 143.8 g sodium carbonate in 400 ml lukewarm water.

After equilibration of the above-described solutions to room temperature, solution B and C are added together. Next, solution A is added. Demineralised water is added to obtain a final volume of 1 litre. This reagens can be used for a couple of days.

Procedure:

1. Prepare a 1 wt % carbohydrate solution.

2. Pipette 1 ml of the carbohydrate solution (diluted to 0.1% (v/v)) in 2 reaction tubes.

3. Add to the first tube every time 0.5 ml demineralised water and to the second tube 0.5 ml 0.1 N iodine solution and 2 to 3 drops 1 N NaOH. Mix these solutions well and leave them for 15 min. at room temperature.

4. Pipette in all tubes 2 ml copper reagens according to Luff. Mix and place the tubes in a boiling waterbath and heat for 5-10 minutes.

5. A red precipitate indicates the presence of reducing carbohydrates.

1.-14. (canceled)

15. A food product comprising at least 0.2% lysine-rich protein by weight of water, wherein the lysine-rich protein comprises at least 4.5 wt % lysine per gram of protein and wherein at least 20% of the lysine residues are glycosylated as determined by an OPA assay, said food product being essentially free of insoluble divalent cation-complexed lysine-rich protein salt and comprising an amount of dissolved divalent cations that would cause the non-glycosylated form of the lysine-rich protein to precipitate as divalent cation complexed protein salt.

16. The food product according to claim 15 comprising at least 50 wt. % water.

17. The food product according to claim 16 comprising at least 80 wt. % water.

18. The food product according to claim 15, wherein the lysine-rich protein exhibits an enthalpic change during denaturation that is at least 90% of its non-modified counterpart.

19. The food product according to claim 15 comprising at least 5% more dissolved divalent cations than the concentration of dissolved divalent cations at which the non-modified protein would precipitate.

20. The food product according to claim 15 comprising at least 2% dissolved divalent cations by weight of the lysine-rich protein.

21. The food product according to claim 15, wherein the lysine-rich protein is soy protein.

22. The food product according to claim 15, wherein the lysine-rich protein is soy milk.

23. The food product according to claim 15, wherein the lysine-rich protein is whey protein.

24. The food product according to claim 22, wherein the lysine-rich protein is cow's milk or products derived therefrom.

25. The food product according to claim 15, wherein said divalent cation comprises Ca²⁺ and/or Mg²⁺ .

26. A method for increasing the divalent cation binding capability of a protein, said method comprising subjecting the protein to Maillard reaction conditions.

27. The method according to claim 26 comprising:
(a) obtaining a mixture, in a dry or solid state, of a protein and a compound comprising a reducing carbonyl moiety, and
(b) heating the mixture to a temperature of at least 40° C. under an atmosphere of at least 55% relative humidity,
whereby the divalent cation binding capability of the protein is increased.

28. A modified protein product composition containing at least 80% by weight of dry matter of glycosylated lysine-rich protein, said lysine-rich protein containing at least 4.5 wt % lysine per gram of said protein, wherein at least 20% of the lysine residues present in the lysine-rich protein have been glycosylated.
